(19) United States
(12) Patent Application Publication
(10) Pub. No.: US 2014/0121679 A1
(43) Pub. Date: May 1, 2014

(54) ELASTIC BAND LIGATION DEVICE AND METHOD FOR TREATMENT OF HEMORRHOIDS

(71) Applicant: Iain G.M. CLEATOR, Vancouver (CA)

(72) Inventor: Iain G.M. CLEATOR, Vancouver (CA)

(21) Appl. No.: 13/892,804

(22) Filed: May 13, 2013

Related U.S. Application Data
(63) Continuation of application No. 12/232,013, filed on Sep. 9, 2008, now abandoned.

(51) Int. Cl. A61B 17/12

(52) U.S. Cl. CPC A61B 17/12
USPC 606/140

(57) ABSTRACT

Elastic band ligation device for treating hemorrhoids and treatment method are provided. The device includes an inner tubular member for retaining an elastic band over the front end. The inner tubular member is arcuate and is insertable into the rectum of a patient. A plunger in the tubular member when pulled generates a suction for drawing hemorrhoidal tissue into the inner tubular member through the front end. A plastic outer tubular pusher sleeve has an arcuate configuration corresponding to the arcuate inner tubular member to provide a limited friction fit over the inner tubular member. The pusher sleeve has a thumb pusher to allow the outer tubular pusher sleeve to be pushed towards the front end of the inner tubular member and release the elastic band from the front end of the inner tubular member to engage hemorrhoidal tissue extending through the opening in the inner tubular member.

### CROSS-REFERENCE TO RELATED APPLICATION

This application is a continuation of U.S. application Ser. No. 12/232,013, filed Sep. 9, 2008, the entire content of which is hereby incorporated by reference in this application.

### BACKGROUND OF THE INVENTION

The present invention relates to the treatment of hemorrhoids by elastic band ligation (alternatively referred to as rubber band ligation). More specifically the invention provides an elastic band ligation device having an arcuate telescopic tubular assembly that may be accurately positioned in the rectum by a single operator to facilitate application of one or more elastic bands to hemorrhoidal tissue.

Treatment of hemorrhoids by elastic band ligation typically involves placing an elastic band on tissue in the rectum above the area of the hemorrhoid where there is little sensation. The tissue trapped in the band being cut off from its blood supply degenerates and is sloughed, and the elastic band along with the sloughed tissue is passed with bowel motions. More importantly, however, the resulting healing process causes the tissue in the vicinity to become fixed and prolapse of the hemorrhoidal tissue is minimized. Furthermore, the elastic band ligation technique has been found to give relief of hemorrhoidal symptoms.

Many devices exist on the market today utilizing the elastic band ligation technique. Examples are U.S. Pat. No. 5,203,863 to Bidoia, U.S. Pat. No. 5,122,149 to Broome, U.S. Pat. No. 5,158,563 to Cosman and U.S. Pat. No. 5,741,273 to O'Regan. The devices disclosed in U.S. Pat. No. 5,203,863 to Bidoia, U.S. Pat. No. 5,122,149 to Broome, U.S. Pat. No. 5,158,563 to Cosman are generally designed to be used in conjunction with an instrument such as a proctoscope or anoscope to directly see the area to be banded. In some cases, it is necessary to employ an assistant to hold the proctoscope or anoscope. However, the use of these scopes, which are generally larger in diameter than banding apparatus, can cause considerable discomfort to a patient and more specifically to one who is suffering symptoms of hemorrhoids.

The device disclosed in U.S. Pat. No. 5,741,273 to O'Regan relates to an elastic band ligation device for treatment of hemorrhoids that may be used without directly seeing the site for banding. Thus, it may be used without a proctoscope or anoscope or any other type of scope or viewing technique. Therefore, because the device can be inserted into the rectum and positioned appropriately without simultaneous visualization, or the need for any type of scope, the banding procedure performed in this manner causes less discomfort to the patient. This device has a suction device incorporated therein to draw hemorrhoidal tissue into an aperture for banding without requiring a second operator or connection to an aspirator.

Ligation is typically performed by initially inspecting the site through a small scope such as a sigmoidoscope, anoscope, proctoscope or other type of scope and making a mental note as to the area where the band should optimally be placed. The scope is then removed and the ligation device is placed in the rectum.

An alternative application is to visualize the area with an anoscope and insert the banding through the lumen of the anoscope and band directly. This can be done if there no anal spasm or fissure as well as hemorrhoids. This is known as direct viewing.

At first, the front end of the device is inserted well past the site for banding. Then, the device is gently withdrawn while it is angled acutely to point in the direction of the site. As the device is being withdrawn, marks on its surface appear at the anal verge which can be used as a guide to the level where the band should optimally be placed in the rectum.

However, it has been found that the device of U.S. Pat. No. 5,741,273 suffers from the drawback that the tip does not engage at the correct angle. As a result, even when bands are placed by direct viewing, re-examination often reveals that the band may be as much as 45 degrees off in one direction or another as far as the circumference of the rectum is concerned. A need exists therefore for an improved elastic band ligation device which engages at the correct angle. The present invention seeks to satisfy that need.

### BRIEF DESCRIPTION OF THE INVENTION

The present invention provides an elastic band ligation device for treatment of hemorrhoidal tissue which is configured to facilitate engagement with hemorrhoidal tissue at the correct angle in the rectum. In one embodiment, the device comprises an inner tubular member having a front end and a rear end, for retaining a stretched elastic band over the front end thereof, the front end having an opening, the inner tubular member having an arcuate configuration extending along at least a portion of the length thereof for a sufficient distance for insertion into the rectum of a patient, a plunger in the tubular member with a handle means extending away from the front end of the inner tubular member, the handle means for sliding the plunger away from the front end of the inner tubular member to provide a suction for drawing hemorrhoidal tissue into the inner tubular member through the opening at the front end, and an outer tubular pusher sleeve having an arcuate configuration corresponding to the arcuate configuration of the inner tubular member to provide a limited friction fit over the inner tubular member, with an external end of the outer tubular pusher sleeve adjacent the stretched elastic band, and having an opposite end of the outer tubular pusher sleeve provided with thumb push means for an operator to push the outer tubular pusher sleeve towards the front end of the inner tubular member and release the elastic band from the front end of the inner tubular member to engage hemorrhoidal tissue extending through the opening in the inner tubular member.

The term “a limited friction fit” a used herein means that the outer tubular pusher sleeve exerts sufficient friction against the inner tubular member such that the two component do not freely slide with respect to each other but require gentle hand-applied pressure to move one relative to the other.

In one embodiment, the ligation device is made of plastic and is disposable. Thus, once it has been used it is discarded and does not have to be sterilized. In another embodiment, the ligation device is made of metal and may be sterilized after each use.

The disposable elastic band ligation device of the invention may be used in a doctor's office and does not generally require any form of anesthetic. One to three elastic bands are generally placed at each patient visit and as many as six bands may be required in total, particularly in advanced cases.

In a further embodiment, the ligation device of the invention may be provided with a separable elastic band introducer device which facilitates introduction of the elastic band(s) onto the front end of the device. In one embodiment, the introducer comprises a front tapered section, a rear section dimensioned such that it can be connected to the front end of the inner tubular member, and a center section disposed between the front and rear sections having an external dimension corresponding to that of the front end of the inner tubular member. In use, the rear end of the introducer is connected to the front end of the inner tubular member and an elastic band is rolled over the tapered front section and over the central section onto the front end of the inner tubular member. Once the band has been placed on the front end of the inner tubular member, the introducer is removed prior to use of the ligation device.

An additional feature is the use, in the plastic version of the ligation device, of a plastic retention fixed ring on the inside of the proximal end of the inner tubular member for preventing the plunger from being withdrawn from the inner tubular member and thus preventing the possibility of the instrument being cleaned and reused. Reuse of the device may be possible when the ligator is fabricated from metal, such as stainless steel, which can be sterilized between uses.

A further additional feature, again in connection with the plastic version of the ligation device, is the use of a treated slightly roughened inside plastic or a material such as Plexiglas SL7 on the inside of the inner tubular member which turns opaque after attempted cleaning and, thus, provides a visual guide that the ligator has been deployed in one patient and must not be reused.

A yet further feature of the invention resides in a method of treating hemorrhoidal tissue in a patient in need of such treatment. The method comprises the steps of: (a) providing an elastic band ligation device of the invention; b) applying an elastic band over the front end of the inner tubular member; (c) inserting the device carrying the elastic band into the rectum of a patient; (d) adjusting the position of the arcuate section to bring the opening into direct alignment with the hemorrhoidal tissue; (e) withdrawing the plunger to exert suction on said hemorrhoidal tissue and draw it through the opening into the inner tubular member; and (f) moving the outer tubular pusher sleeve against the elastic band to urge the elastic band onto the hemorrhoidal tissue.

An additional aspect of the invention provides a non-latex elastic band for use in the treatment of patients who exhibit or may exhibit a latex allergic reaction.

### BRIEF DESCRIPTION OF THE DRAWINGS

FIG. 1 is a side view of a first embodiment of ligation device of the present invention showing an elastic band introducer device aligned with the distal end;

FIG. 2 is an end view of the ligation device of FIG. 1 from the proximal end;

FIG. 3 is a view from the underside of the ligation device of FIG. 1;

FIG. 4 is a cross-sectional view of the ligation device of FIG. 3 along the line A-A;

FIG. 5 is a perspective view of the ligation device of FIG. 1;

FIG. 6 is a side view of a second embodiment of a ligation device of the present invention showing an elastic band introducer device aligned with the distal end;

FIG. 7 is an end view of the ligation device of FIG. 6;

FIG. 8 is a view from the underside of the ligation device of FIG. 6;

FIG. 9 is a cross-sectional view of the ligation device of FIG. 8 along the line B-B;

FIG. 10 is a perspective view of the ligation device of FIG. 6.

FIGS. 11 and 12 are cross-sectional views of the human anatomy showing, respectively, the use of the ligation device of the present invention and the prior art straight ligation device;

FIGS. 13a, 13b, 13c and 13d show a non-latex elastic band which may be employed with the ligation device of the invention.

### DETAILED DESCRIPTION OF THE INVENTION

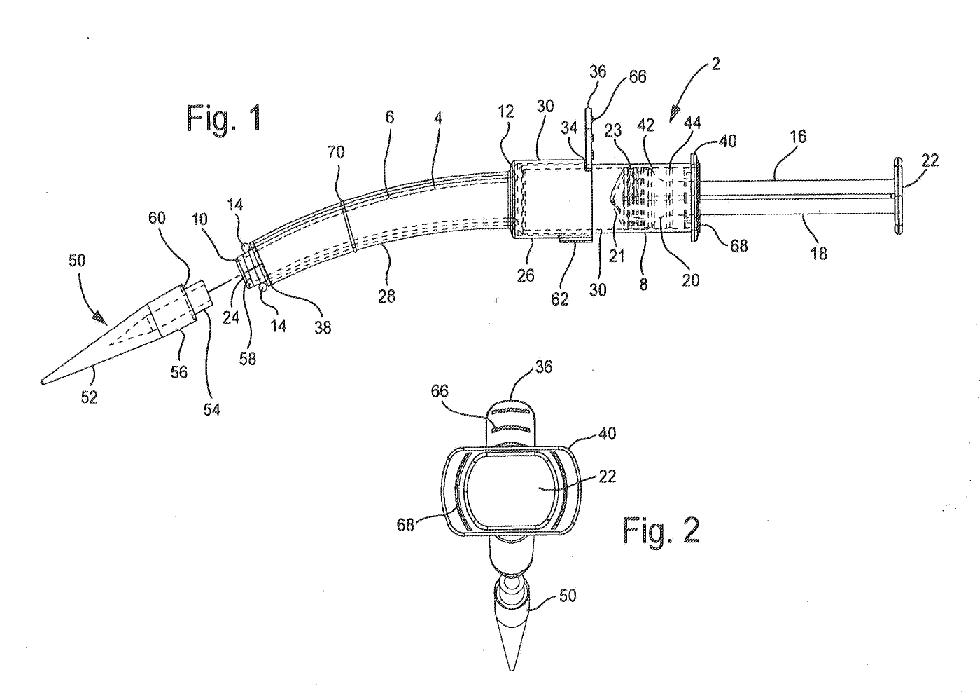

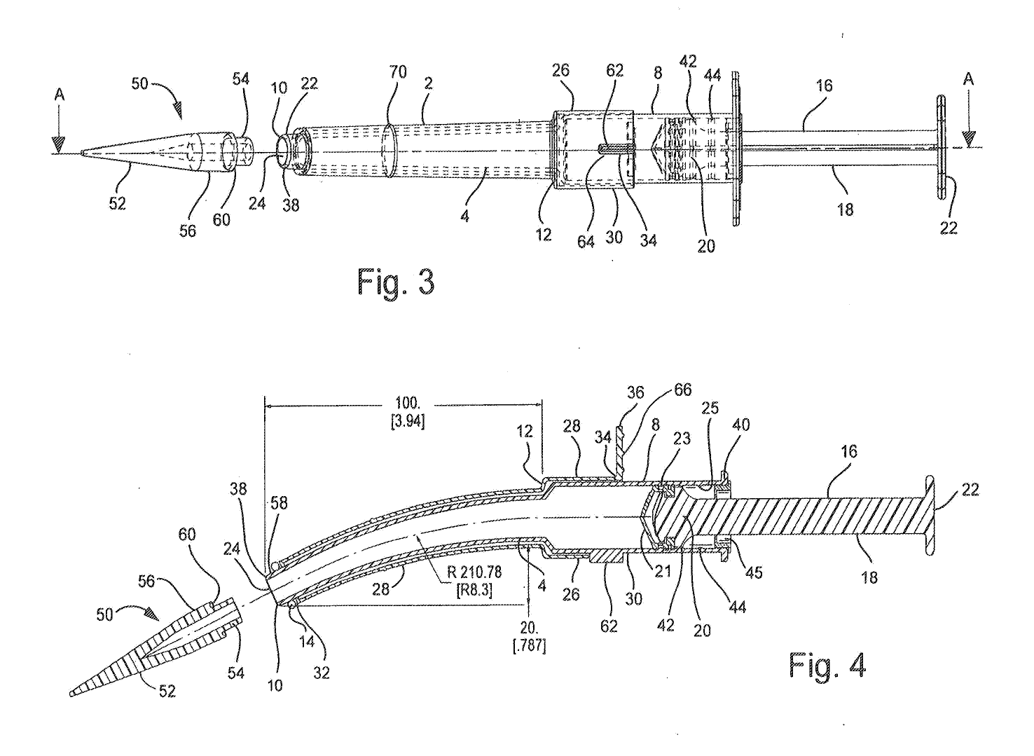

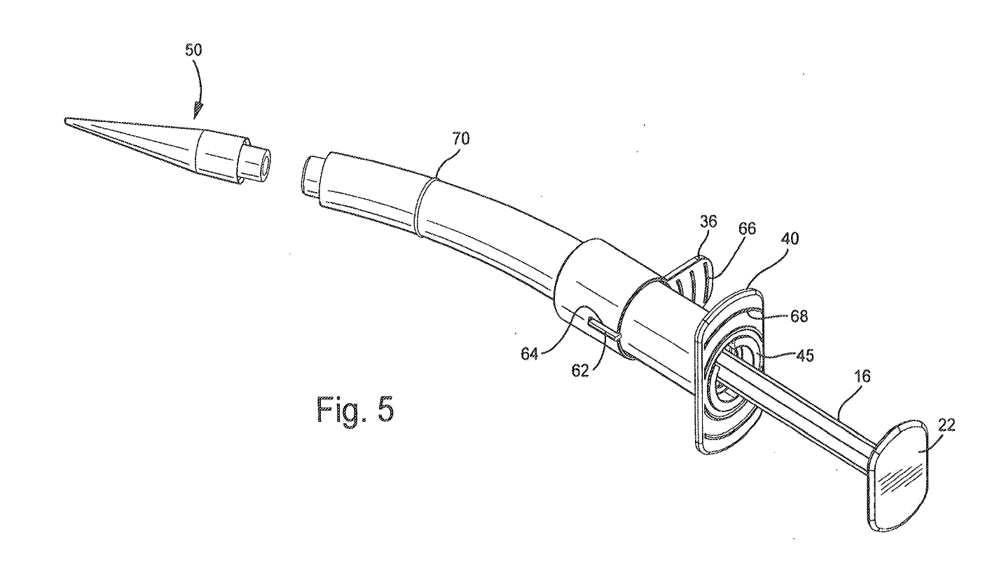

Referring to the drawings, FIGS. 1-5 show a first embodiment of a ligation device of the invention, generally referenced 2, comprising a plastic inner tubular member 4 having an arcuate section 6 and an integrally formed straight cylindrical section 8. The arcuate section 6 has a front end 10 and a rear end 12, for retaining a stretched elastic band 14 over the front end 10 thereof.

In the embodiment illustrated in FIGS. 1-5, the inner tubular member 4 has an arcuate configuration extending continuously from the rear end 12 to the front end 10 and for a sufficient distance to facilitate insertion into the rectum of a patient. The radius of the arc is generally in the range of about 200-215 mm, for example about 210-211 mm. The embodiment shown in FIG. 4 has an arc radius of 210.78 mm (8.3″). The distance that the arcuate section extends from the tip of the front end 10 to the rear end 12 of the arcuate section generally ranges from about 85-115 mm, for example about 95-105 mm. In the embodiment shown in FIG. 4, this distance is 100 mm (3.94″). The vertical distance from the lower portion of the front end 10 to the lower surface of the rear end 12 is generally in the range of about 15-25 mm, for example 18-22 mm. In the embodiment shown in FIG. 4B, this distance is 20 mm (0.787″).

A plunger 16 is slideably disposed in the cylindrical section 8, and has a stem center section 18, a head 20 at one end of a stem section 18 and a handle 22 at the other end of the stem section 18 and extending away from the front end 10 of the inner tubular member 4. The head 20 is typically fabricated from rubber and is adapted for sealingly engaging with an inner wall of the cylindrical section 8. In the embodiment shown, a sealing member 21 is mounted on the head 20 for sealingly engaging at 23 with the inner wall 25 of the cylindrical section 8.

The handle 22 permits the plunger 16 to be slid away from the front end 10 of the inner tubular member 4 to provide a suction for drawing hemorrhoidal tissue into the inner tubular member 4 through the opening 24 at the front end 10. The opening 24 may be slightly smaller in diameter than the inner tubular member 4. In another embodiment, the cylindrical section 8 and inner tubular member 4 may be separate components joined together.

A plastic outer tubular pusher sleeve 26 is provided having an arcuate section 28 corresponding to the arcuate configuration of the inner tubular member 4 and a straight cylindrical section 30. The outer tubular pusher sleeve 26 has a limited friction fit over the length of the inner tubular member 4, and has an external end 32 adjacent the stretched elastic band 14 and an opposite end 34 provided with thumb push means 36 for an operator to push the outer tubular pusher sleeve 26 towards the front end 10 of the inner tubular member 4 and release the elastic band 14 from the front end 10 of the inner tubular member 4 to engage hemorrhoidal tissue extending through the opening 24 in the inner tubular member 4.

A circumferential ring marker 70 is provided on the external circumference of the outer tubular pusher sleeve 26. This serves as a depth marker to the user with regard to the extent of insertion of the device into the rectum of the patient.

The inner tubular member 4 is provided with a guide protrusion 62 which extends radially outwardly from cylindrical section 8. This guide protrusion slidingly engages with a slot 64 formed in the opposite end 34 of the pusher sleeve 26 which prevents relative rotation of the inner tubular member 4 and the pusher sleeve 26 as the pusher sleeve 26 is moved longitudinally relative to the inner tubular member 4.

The inner tubular member 4 may be provided with a rounded, bevelled or radiused front end 38 to further assist in introduction into the rectum of the patient and also to facilitate ready departure of the elastic band 14 off the front end 10 onto the hemorrhoidal tissue upon actuation of the pusher sleeve 26. In addition, the inner tubular member 4 may be slightly tapered increasing in diameter as it extends back from the front end 10 to the rear end 12. In another embodiment, the inner tubular member 4 may be cylindrical in shape with no longitudinal taper.

The cylindrical section 8 of the inner tubular member 4 is substantially cylindrical along its entire length and is not tapered. An outside shoulder 40 allows the cylindrical section 8 to be gripped when the plunger 16 is pulled back.

A first shoulder ring 42 is located towards the distal end of the cylindrical section 8 and acts as a locking system so that when the plunger 16 is pulled back and the head 20 rides over the first shoulder ring 42 it is held in that position and holds a vacuum in the cylindrical section 8 and the inner tubular member 4. If there was no locking system, the plunger 16 would immediately return towards the distal end to its original position and no vacuum would remain to pull the hemorrhoidal tissue into the inner tubular member 4. A second shoulder ring 44 is positioned further along from the first shoulder ring 42 towards the proximal end and acts as a stop to prevent the plunger 16 from being pulled out of the cylindrical section 8. Whereas shoulders 42, 44 are shown for locking the plunger 16 in the cylindrical section 8, it will be apparent to those skilled in the art that other types of clamping systems to stop the plunger 16 returning in the cylindrical section 8 may be provided.

A plastic retention fixed ring 45 is provided in the proximal end of the inner tubular member 4 for preventing the plunger 16 from being withdrawn from the inner tubular member 4 and thus preventing the possibility of the instrument being cleaned and reused. Reuse of the device may be possible when the ligator is fabricated from metal such as stainless steel which can be sterilized between uses.

A further feature, again in connection with the plastic version of the ligation device, is the use of a treated slightly roughened inside plastic or similar material on the inside of the inner tubular member which turns opaque after attempted cleaning and, thus, provides a visual guide that the ligator has been deployed in one patient and must not be reused. An example of such material is Plexiglas SL7.

In a further embodiment, there is provided a separable cone-shaped elastic band introducer device 50 which facilitates introduction of elastic band(s) 14 onto the front end 10 of the device 2. In one embodiment, the introducer 50 comprises a tapered front section 52, a rear straight cylindrical section 54 dimensioned such that it can be connected to the front end 10 of the inner tubular member 4, typically by insertion into the opening 24, and a center section 56 disposed between the front section 52 and the rear section 54 having an external circumferential dimension corresponding to that of the front end 10 of the inner tubular member 4. A thin sleeve 60 is provided which overlaps the front end 10 of the inner tubular member 4 and prevents the elastic band 14 from becoming lodged in the join between the introducer 50 and the inner tubular member 4.

In use of the introducer, the rear end 54 of the introducer 50 is connected to the front end 10 of the inner tubular member 4 by insertion into the opening 24, and an elastic band 14 is rolled over the tapered front section 52 and over the central section 56 onto the front end 10 of the inner tubular member 4. A short section of the front end 10 may be provided with one or more ridges 58 to hold the elastic band 14 in place prior to application to the hemorrhoidal tissue. Once the elastic band 14 has been placed on the front end 10 of the inner tubular member 4, the introducer 50 is removed prior to use of the ligation device.

In operation, once a physician has examined the rectum of a patient and discovered the position of the hemorrhoidal tissue, the scope through which the inspection has initially occurred is removed and the position of the hemorrhoidal tissue is memorized. A stretched elastic band 14, or rubber band, is positioned over the inner tubular member 4 adjacent the front end 10 using the introducer 50 as described above. The introducer 50 is then removed and set aside. The arcuate section 6 of the device bearing the elastic band is inserted carefully into the rectum of the patient to a point beyond the location of the hemorrhoidal tissue to be removed, and then withdrawn until the tissue is adjacent the opening 24, and position is noted with regard to the ring marker 70.

An important advantage of the present device as compared to the prior straight devices is that, because of the curvature of the device, the opening 24 upon insertion of the device to the location of the hemorrhoidal tissue to be banded faces directly towards and contacts the hemorrhoidal tissue so that there is essentially no air gap between the end of the device and the tissue to be drawn into the device, thereby allowing suction to be generated upon withdrawal of the plunger.

This is termed placing the device at the correct “L angle” (see FIGS. 11 and 12, discussed in more detail below where the L angle is shown by the letter “L”). The L angle is defined as follows: the upper limb of the L is the wall of the rectum just above where the hemorrhoid arises. The lining or mucosa is firmly attached to the muscle over the upper limb of the L. The transverse portion of the L is the hemorrhoid itself, and depending on how large it becomes, slides out and down the anal canal to the outside. By placing the ligation device of the present invention exactly to the L angle and then placing suction on the tissue, the loose hemorrhoidal tissue at the transverse limb of the L is drawn up to its correct position, but the tissue from the upper L is fixed and not sucked upwardly. When the elastic band is released (“fired”) from the device, the tissue is tacked up to its correct position because of the firm attachment of the upper limb of the L, and in two days the excess tissue falls off. An additional advantage is that this L angle area is free from pain for the patient as there are no pain fibers in that location, unlike the positioning of the early metal banders which apply the bands lower down. The arcuate or curved feature of the ligation device of the present invention facilitates ready identification of the proper location at the L angle and allows for accurate banding at that location, unlike the prior straight predecessor.

FIG. 11 illustrates the situation in which the device of the invention has been inserted into position at the L angle adjacent the hemorrhoidal, with no air gap between the front end of the device 10 and the tissue T to enable suction to be applied to the tissue through the opening 24 upon withdrawal of the plunger. This obviates the need for the operator to move the device any significant amount within the rectum to try and line up the hemorrhoidal tissue with the opening 24, which induces discomfort in the patient and is a disadvantage associated use of the prior straight ligation devices (see FIG. 12). In fact, it is quite difficult to get the prior straight ligation devices to line up exactly with the hemorrhoidal tissue. In such a situation, when the plunger is withdrawn, the straight ligation device may either inflict suction on tissue in the region below the hemorrhoidal tissue which causes pain in the patient or, because it is not directly lined up with the hemorrhoidal tissue, the straight device may simply suck air which results in incomplete capture of the hemorrhoidal tissue.

With the device of the present invention, when the plunger 16 is withdrawn past the first shoulder ring 42 to form a vacuum, because the opening 24 is directly lined up with and contiguous with the hemorrhoidal tissue, the entire of hemorrhoidal tissue is drawn into the inner tubular member 4 through the opening 24. The operator then releases the plunger 16 and pushes the outer plastic pusher sleeve 26 forward with the push means 36 so that the stretched elastic band 14 is pushed off the front end 10 of the inner tubular member 4 to capture the hemorrhoidal tissue. The ligation device can then be removed and the procedure may be carried out at least three further times during one patient's visit to the doctor's office. No aspirator or other device need be attached to the ligation device as the built-in plunger mechanism sucks the hemorrhoidal tissue into the inner tubular member 4 and holds it there while the operator introduces each elastic band into surrounding engagement with the hemorrhoidal tissue to be removed. The shoulder 40 and the push means 36 may be provided with groves or protuberances 66, 68 to increase friction between the push means 36 and the thumb or finger(s) of the user.

The ligation device 2 may be made in different sizes, and 8 mm, 10 mm and 12 mm inside diameters of the inner tubular member 4 are generally preferred sizes. The dimension of the opening 24 is generally about 0.5 to 1 mm less than the internal diameter of the inner tubular member 4 to permit radiused rounded edges at the front end 10.

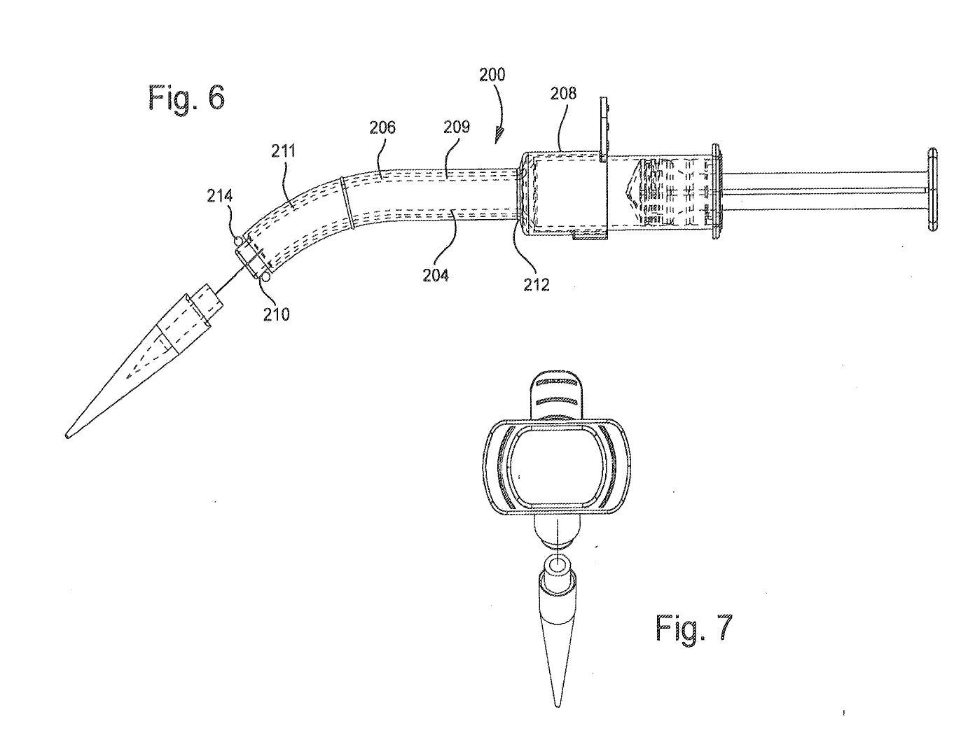

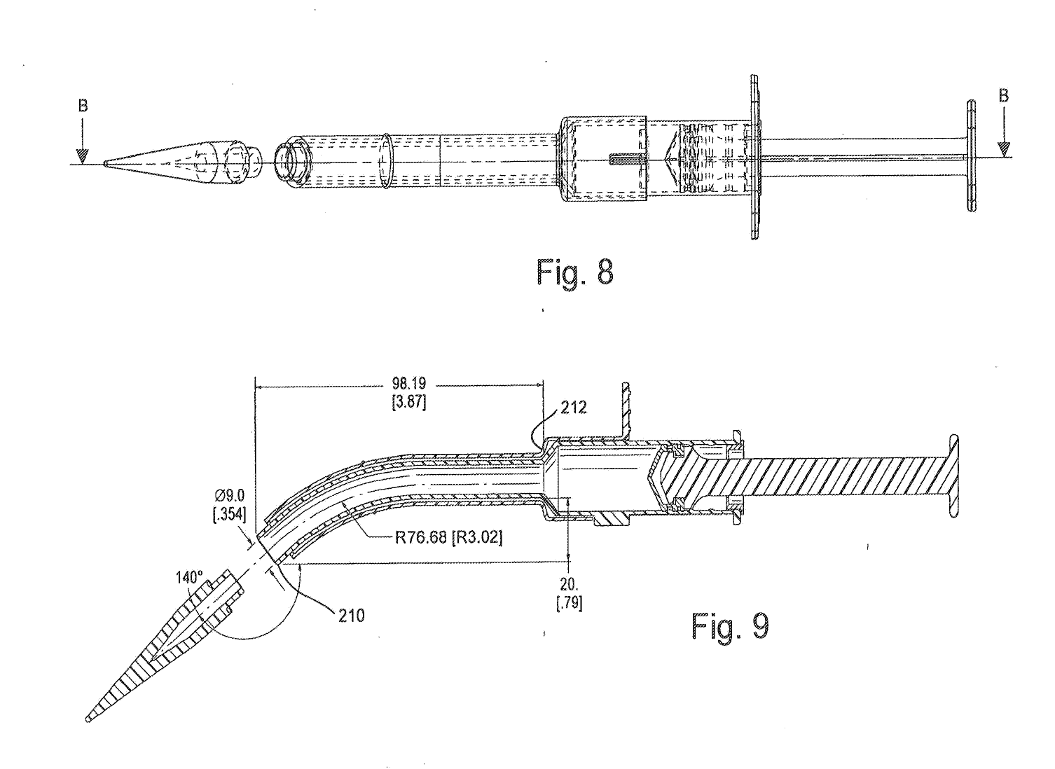

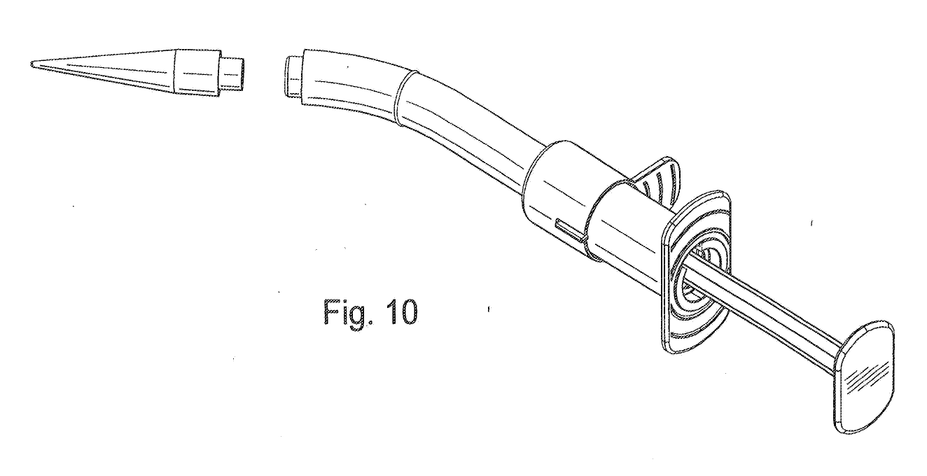

Referring to FIGS. 6-10, there is shown an alternative embodiment of a ligation device of the invention, in which many of the components are similar to those described with regard to the device of FIGS. 1-5 and are numbered similarly beginning with 200. Features notable with respect to the device of FIGS. 6-10 will be described in more detail below.

The ligation device shown in FIGS. 6-10, generally referenced 200, comprises a plastic inner tubular member 204 having a section 206, a portion 209 of which is straight and a portion 211 of which has an arcuate configuration. The section 206 is integrally formed with a straight cylindrical section 208. The arcuate section 211 has a front end 210 and a rear end 12, the front end 210 for retaining a stretched elastic band 214 thereover.

In the embodiment illustrated in FIGS. 6-10, the inner tubular member 204 which extends for a distance sufficient to facilitate insertion into the rectum of a patient. As seen in FIG. 9, the angle formed between the longitudinal axis of the straight section 209 and the axis defined by the end of the curved section 211 is about 140 degrees. The radius of the arc between the straight section 209 and the axis defined by the end of the curved section 211 is generally in the range of about 75-78 mm, for example 76.68 mm (3.02 inches). The distance from the tip of the front end 210 to the rear end 212 is generally in the range of about 90-115 mm, for example about 95-99 mm. In the embodiment shown in FIG. 9, this length is 98.19 mm (3.87″). The vertical distance from the lower portion of the front end 210 to the lower surface of the rear end 212 is generally in the range of about 15-25 mm, for example about 18-22 mm. In the embodiment shown in FIG. 9, this distance is 20 mm (0.79″).

The ligation device of the invention may be made of plastic, either clear or opaque plastic, and may be disposed after use. Alternatively, the ligation device of the invention may be fabricated from stainless steel and may be sterilized after each use.

The elastic band ligation device of the invention designed for use by one person and is integrated with its own suction generation mechanism and rubber band applicator mechanism. The ligator is specially configured to engage the upper end of the hemorrhoid at an optimal angle for applying suction to the hemorrhoid and aspirating the tissue into the ligator without escape of suction. The device is also configured such that ligation occurs at a point where the hemorrhoid can be banded without pain to the patient and where suction of hemorrhoid and the tacking effect of the band to prevent prolapse are optimal. In addition, the device has an overall diameter which is smaller than other devices which employ banding and can be used with or without an anoscope, more usually without an anoscope thereby reducing the level of discomfort to the patient. The ligator is typically fabricated from plastic and may be provided with a plastic sleeve to prevent washing and reuse. In addition, or alternatively, the device may be fabricated from a material that turns opaque after use and/or after attempted cleansing to prevent reuse.

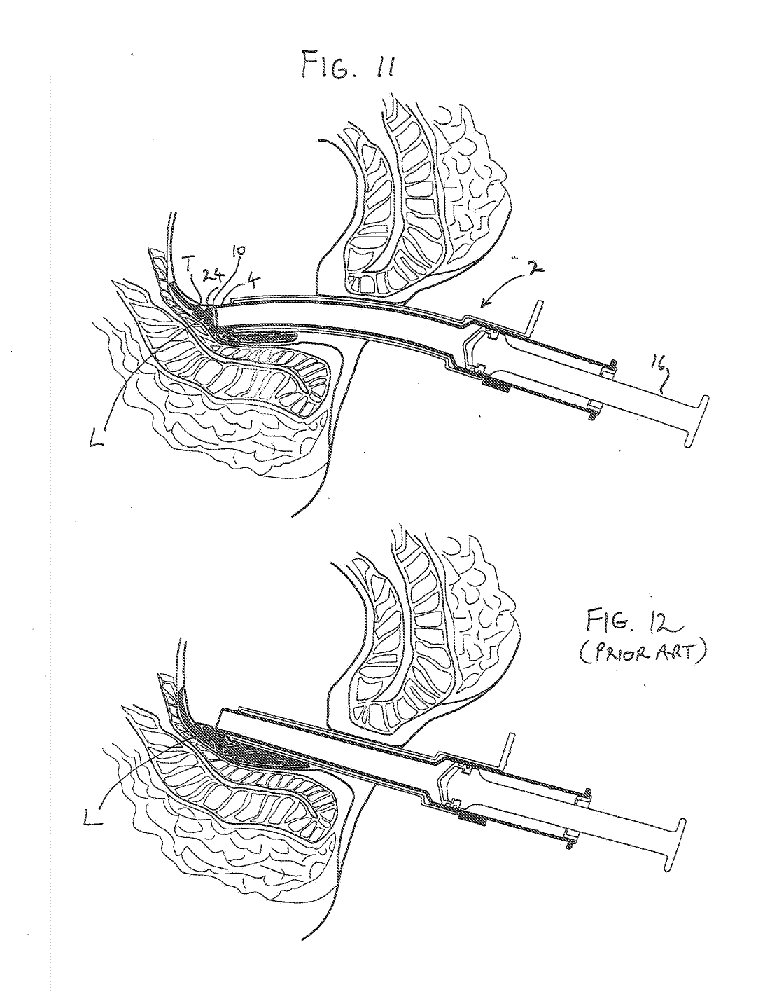

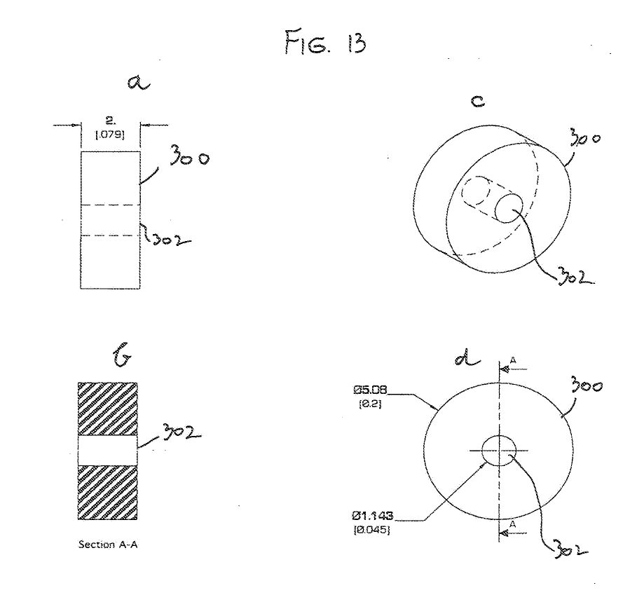

In the embodiment shown in FIG. 1, the band 14 is an O-ring, is circular in cross-section and is typically fabricated from rubber or latex material. In an alternative embodiment, the band may be fabricated from a non-latex material which is desirable in those instances where the patient is or may be subject to a latex allergic reaction. An example of a non-latex band is illustrated in FIGS. 13a-13d which shows a cylindrical shaped band 300 with a central aperture 302 and a non-circular, typically square, cross-section. Typically, for the device of the invention, the diameter of the band is in the range of 5-6 mm, for example about 5.08 mm (0.2 inches). The diameter of the aperture 302 is typically in the range of 1-1.5 mm for example about 1.143 mm (0.045 inches). The band 300 is applied to the device in the same way as the rubber band discussed earlier, namely through use of the introducer.

While the invention has been described in connection with what is presently considered to be the most practical and preferred embodiment, it is to be understood that the invention is not to be limited to the disclosed embodiment, but on the contrary, is intended to cover various modifications and equivalent arrangements included within the spirit and scope of the appended claims.

What is claimed is:

1. An elastic band ligation device for treatment of hemorrhoidal tissue, comprising:
an inner tubular member having a front end and a rear end, for retaining a stretched elastic band over the front end thereof, the front end having a opening, the inner tubular member having an arcuate section extending along at least a portion of the length thereof for a sufficient distance for insertion into the rectum of a patient,
a plunger in the tubular member with a handle means extending away from the front end of the inner tubular member, the handle means for sliding the plunger away from the front end of the inner tubular member to provide a suction for drawing hemorrhoidal tissue into the inner tubular member through the opening at the front end, and
a plastic outer tubular pusher sleeve having an arcuate section corresponding to the arcuate section of the inner tubular member to provide a limited friction fit over the inner tubular member, with an external end of the outer tubular pusher sleeve adjacent the stretched elastic band, and having an opposite end of the outer tubular pusher sleeve provided with means for an operator to push the outer tubular pusher sleeve towards the front end of the inner tubular member and release the elastic band from the front end of the inner tubular member to engage hemorrhoidal tissue extending through the opening in the inner tubular member.

2. The elastic band ligation device according to claim 1 wherein the arcuate section of the inner tubular member extends continuously from the rear end to the front end and has a radius in the range of about 200-215 mm.

3. The elastic band ligation device according to claim 2 wherein arc radius is about 210-211 mm.

4. The elastic band ligation device according to claim 2 wherein arc radius is about 210.78 mm (8.3″).

5. The elastic band ligation device according to claim 1 wherein the length from the front end to the rear end is in the range of about 85-115 mm.

6. The elastic band ligation device according to claim 1 wherein the length from the front end to the rear end is about 95-105 mm.

7. The elastic band ligation device according to claim 1 wherein the length from the front end to the rear end is 100 mm (3.94″).

8. The elastic band ligation device according to claim 1 wherein the vertical distance from the front end to the rear end is in the range of about 15-25 mm.

9. The elastic band ligation device according to claim 1 wherein the vertical distance from the front end to the rear end is about 18-22 mm.

10. The elastic band ligation device according to claim 1 wherein the vertical distance from the front end to the rear end is 20 mm (0.787″).

11. The elastic band ligation device according to claim 1 and further including a separable elastic band introducer for introduction of elastic band(s) onto the front end.

12. The elastic band ligation device according to claim 11 wherein the introducer device comprises a front tapered section, a rear section dimensioned such that it can be connected to the front end of the inner tubular member, and a center section disposed between the front and rear sections having an external dimension corresponding to that of the front end of the inner tubular member.

13. The elastic band ligation device according to claim 1 wherein the tubular inner member has a first proximal section, a portion of which is straight and a second distal section, a portion of which has an curved configuration.

14. The elastic band ligation device according to claim 13 wherein the angle formed between a longitudinal axis of the straight section and the axis defined by the end of the curved section is about 135-145 degrees.

15. The elastic band ligation device according to claim 14 wherein the angle formed between a longitudinal axis of the straight section and the axis defined by the end of the curved section is about 140 degrees.

16. The elastic band ligation device according to claim 14 wherein the radius of the arc is generally in the range of about 75-78 mm.

17. The elastic band ligation device according to claim 15 wherein arc radius is about 76.68 mm (3.02″).

18. The elastic band ligation device according to claim 13 wherein the length from the tip of the front end to the rear end is in the range of about 90-115 mm.

19. The elastic band ligation device according to claim 13 wherein the length from the tip of the front end to the rear end is about 95-99 mm.

20. The elastic band ligation device according to claim 19 wherein the length from the tip of the front end to the rear end is 98.19 mm (3.87″).

21. The elastic band ligation device according to claim 13 wherein the vertical distance from the front end to the rear end is in the range of about 15-25 mm.

22. The elastic band ligation device according to claim 13 wherein the vertical distance from the front end to the rear end is about 18-22 mm.

23. The elastic band ligation device according to claim 22 wherein the vertical distance from the front end to the rear end is 20 mm (0.79″).

24. The elastic band ligation device according to claim 1 wherein the inner tubular member and the outer tubular pusher sleeve have a taper with the diameter increasing from the front end.

25. The elastic band ligation device according to claim 1 wherein the inner tubular member includes a cylindrical plunger section spaced from the front end of the inner tubular member, the plunger sliding in the plunger section to provide a suction therein.

26. The elastic band ligation device according to claim 10 wherein the introducer has a thin sleeve that overlaps the front end of the inner tubular member.

27. The elastic band ligation device according to claim 1 wherein the outer tubular pusher sleeve has a slot extending parallel to a sleeve axis.

28. The elastic band ligation device according to claim 1 wherein the inner tubular member and the outer tubular pusher sleeve are constructed of stainless steel.

29. The elastic band ligation device according to claim 1 wherein the inner tubular member and the outer tubular pusher sleeve are constructed of plastic material.

30. The elastic band ligation device according to claim 1 wherein the pusher sleeve has a depth marker thereon to aid in positioning the device in the rectum of a patient.

31. The elastic band ligation device according to claim 1 wherein the front end of the inner tubular member is radiused.

32. The elastic band ligation device according to claim 1 including locking means to hold the plunger in a pulled back position in the tubular member to retain a suction in the inner tubular member.

33. The elastic band ligation device according to claim 32 wherein the locking means is at least one shoulder ring in the tubular member to prevent the plunger from moving and maintain suction at two different pressures depending on the ring engaged, thereby enabling more hemorrhoid material to be banded where indicated by size.

34. The elastic band ligation device according to claim 1 wherein the arcuate section orients the opening to directly engage the upper end of the hemorrhoid at an angle which permits application of suction to the hemorrhoid and aspiration of the tissue into the ligator without loss of suction.

35. The elastic band ligation device according to claim 1 wherein the arcuate section is configured such that ligation occurs at a point where the hemorrhoid is banded without inflicting pain to the patient and wherein suction of hemorrhoid and tacking of the band to the hemorrhoid prevent prolapse.

36. The elastic band ligation device according to claim 1 wherein the inner tubular member has a diameter such that the device can be used without an anoscope thereby reducing discomfort to the patient.

37. The elastic band ligation device according to claim 1 wherein the ligator is fabricated from plastic material and has a plastic sleeve to prevent washing and reuse.

38. The elastic band ligation device according to claim 1 wherein the ligator is fabricated from a material that turns opaque after use and/or after attempted cleansing to prevent reuse.

39. The elastic band ligation device according to claim 1 wherein the elastic band is a non-latex elastic band of non-circular cross-section.

40. A method of treating hemorrhoidal tissue in a patient in need of such treatment, said method comprising the steps of:
(a) providing an elastic band ligation device comprising an inner tubular member having a front end and a rear end, for retaining a stretched elastic band over the front end thereof, the front end having a opening, the inner tubular member having an arcuate section extending along at least a portion of the length thereof for a sufficient distance for insertion into the rectum of a patient, a plunger in the tubular member with a handle means extending away from the front end of the inner tubular member, the handle means for sliding the plunger away from the front end of the inner tubular member to provide a suction for drawing hemorrhoidal tissue into the inner tubular member through the opening at the front end, and a plastic outer tubular pusher sleeve having an arcuate section corresponding to the arcuate section of the inner tubular member to provide a limited friction fit over the inner tubular member, with an external end of the outer tubular pusher sleeve adjacent the stretched elastic band, and having an opposite end of the outer tubular pusher sleeve provided with thumb push means for an operator to push the outer tubular pusher sleeve towards the front end of the inner tubular member and release the elastic band from the front end of the inner tubular member to engage hemorrhoidal tissue extending through the opening in the inner tubular member;
(b) applying an elastic band over said front end of said inner tubular member;
(c) inserting said device carrying said elastic band into the rectum of a patient;
(d) adjusting the position of the arcuate section to bring said opening into direct alignment with said hemorrhoidal tissue;
(e) withdrawing said plunger to exert suction on said hemorrhoidal tissue and draw it through said opening into said inner tubular member; and
(f) moving said outer tubular pusher sleeve against said elastic band to urge said elastic band onto said hemorrhoidal tissue.

41. Method according to claim 39 wherein the ligation device is used on its own or together with an anoscope.

42. Method according to claim 39 wherein the ligation device is placed at the L angle to facilitate effective sealing of the front end of the ligation device at the correct location on the hemorrhoid.
